(19) United States
(12) Patent Application Publication
(10) Pub. No.: US 2014/0072966 A1
(43) Pub. Date: Mar. 13, 2014

(54) GENETIC BIOMARKERS FOR GLUCOSE-6-PHOSPHATE DEHYDROGENASE DEFICIENCY

(71) Applicants: MAMDOOH ABDULLAH GARI, JEDDAH (SA); ASHRAF RESIQ DALLOL, JEDDAH (SA); HUDA ABDULRAHMAN BANNI, JEDDAH (SA); ADEL MOHAMMED ABUZENADAH, JEDDAH (SA); ADEEL GULAZAR CHAUDHARY, JEDDAH (SA); MOHAMMED HUSSEIN AL-QAHTANI, JEDDAH (SA); WAEL ZAKI KAFIENAH, BRISTOL (GB); JEFFREY BIDWELL, BRISTOL (GB)

(72) Inventors: MAMDOOH ABDULLAH GARI, JEDDAH (SA); ASHRAF RESIQ DALLOL, JEDDAH (SA); HUDA ABDULRAHMAN BANNI, JEDDAH (SA); ADEL MOHAMMED ABUZENADAH, JEDDAH (SA); ADEEL GULAZAR CHAUDHARY, JEDDAH (SA); MOHAMMED HUSSEIN AL-QAHTANI, JEDDAH (SA); WAEL ZAKI KAFIENAH, BRISTOL (GB); JEFFREY BIDWELL, BRISTOL (GB)

(73) Assignees: UNIVERSITY OF BRISTOL, BRISTOL (GB); KING ABDULAZIZ UNIVERSITY, JEDDAH (SA)

(21) Appl. No.: 13/607,647

(22) Filed: Sep. 7, 2012

(51) Int. Cl. C12Q 1/68; C07H 21/04

(52) U.S. Cl. 435/6.11; 536/23.2

(57) ABSTRACT

The genetic biomarkers for glucose-6-phosphate dehydrogenase (G6PD) deficiency are five haplotypes representing mutations of the G6PD gene on the X chromosome. Each of these haplotypes codes for more than one non-conservative amino acid change. The present inventors have discovered that when mutations of the G6PD gene result in at least two non-conservative amino acid changes in combination, expression of the G6PD enzyme or the stability of the G6PD enzyme is severely decreased, presenting a substantial risk of disease resulting from the deficiency, even in female patients who would normally be considered asymptomatic carriers of the genetic mutation(s).

The Applicants hereby incorporate by reference the sequence listing contained in the ASCII text file titled 30287_{—}20_ST25.txt, created Dec. 2, 2011 and having 2.89 KB of data (3.00 KB on disk),

### BACKGROUND OF THE INVENTION

1. Field of the Invention

The present invention relates to the genetic biomarkers and their use in the prevention, diagnosis, and treatment of disease, and particularly to genetic biomarkers for glucose-6-phosphate dehydrogenase deficiency that provide for identifying patients particularly susceptible to hemolytic anemia, neonatal jaundice, and other conditions when the gene encoding the enzyme contains certain single nucleotide polymorphisms resulting from mutation of the gene.

2. Description of the Related Art

The glucose-6-phosphate dehydrogenase (G6PD) gene encodes the enzyme glucose-6-phosphate dehydrogenase. The enzyme is involved in the normal processing of carbohydrates and plays a critical role in red blood cells. It is responsible for the first step in the pentose phosphate cycle, a pathway that converts glucose to ribose-5-phosphate, which is the building block of purines and pyrimidines. G6PD catalyzes the production of NADPH, which plays a major role in protecting cells from potentially harmful reactive oxygen species.

G6PD deficiency is a very common human metabolic inborn error that affects more than 400 million people worldwide. Diseases caused by deficiency in the G6PD enzyme are statistically distributed with the highest frequency in Africa, Asia, the Mediterranean, and the Middle East. GOD deficiency is caused by mutations in the GOD gene on chromosome X. These mutations lead to functional variants of proteins, resulting in different biochemical and clinical phenotypes. The most common clinical manifestations are neonatal jaundice and acute hemolytic anemia, which is triggered by an exogenous agent in most patients that suffer from the affliction. In some cases, the neonatal jaundice is severe enough to cause death or permanent neurological damage. In a proportion of cases, these manifestations may be life threatening, but fortunately, apart from episodes of hemolytic anemia, most G6PD-deficient individuals are usually asymptomatic. A very small proportion of G6PD-deficient individuals have chronic hemolytic anemia, which can be severe. However, total loss of G6PD activity is fatal. Because the disorder has an X-linked recessive mode of inheritance, males are usually more severely affected than females, although homozygosity, compound heterozygosity, or skewed X-inactivation of affected chromosomes may produce symptoms in females.

There are over 190 recorded G6PD gene mutations. However, only a few previously published reports of G6PD phenotype-genotype correlations have identified genotypes that represent combinations of more than one non-conservative mutation. Moreover, the previously disclosed methods cannot predict the severity of the deficiency. As with any disease, early detection of persons subject to G6PD deficiency, and particularly in a form likely to produce the most severe debilitating effects, is key to treating and ameliorating the effects of genetically induced deficiency of the enzyme.

Thus, genetic biomarkers for glucose-6-phosphate dehydrogenase deficiency solving the aforementioned problems is desired.

### SUMMARY OF THE INVENTION

The genetic biomarkers for glucose-6-phosphate dehydrogenase (G6PD) deficiency are five haplotypes representing mutations of the G6PD gene on the X chromosome. Each of these haplotypes codes for more than one non-conservative amino acid change. The present inventors have discovered that when mutations of the G6PD gene result in at least two non-conservative amino acid changes in combination, expression of the G6PD enzyme or the stability of the G6PD enzyme is severely decreased, presenting a substantial risk of disease resulting from the deficiency, even in female patients who would normally be considered asymptomatic carriers of the genetic mutation(s).

The genetic biomarkers for glucose-6-phosphate dehydrogenase (G6PD) deficiency provides a method for screening patients to determine patients who are candidates for developing severe disease (such as hemolytic anemia and neonatal jaundice) resulting from insufficient expression of the G6PD enzyme, opening the door to targeted preventive treatment. The method involves obtaining samples of DNA from a patient and sequencing the G6PD gene on the X chromosome. Identification of at least one haplotype selected from the group consisting of the Jeddah A, Jeddah B, Jeddah C, Jeddah D, and Jeddah E haplotypes is diagnostic for inadequate expression of G6PD enzyme to a degree that represents high risk of severe disease from deficiency of the enzyme. Haplotypes may be identified directly in male patients, who are hemizygous for the X chromosome, and may be identified by PHASE haplotype reconstruction in female patients.

These and other features of the present invention will become readily apparent upon further review of the following specification and drawings.

### BRIEF DESCRIPTION OF THE DRAWINGS

FIG. 1 is a chart showing the correlation between G6PD haplotype and G6PD enzyme levels in males with non-conservative mutations, where expression of the haplotype is dominant.

FIG. 2 shows the correlation between G6PD haplotype and G6PD enzyme levels in females with non-conservative mutations, where expression of the haplotype is mosaic.

FIG. 3 is a table showing haplotypes identified by 6-locus haplotyping.

Similar reference characters denote corresponding features consistently throughout the attached drawings.

### DETAILED DESCRIPTION OF THE PREFERRED EMBODIMENTS

The genetic biomarkers for glucose-6-phosphate dehydrogenase (G6PD) deficiency are five haplotypes representing mutations of the G6PD gene on the X chromosome. Each of these haplotypes codes for more than one non-conservative amino acid change. The present inventors have discovered that when mutations of the G6PD gene result in at least two non-conservative amino acid changes in combination, expression of the G6PD enzyme or the stability of the G6PD enzyme is severely decreased, presenting a substantial risk of disease resulting from the deficiency, even in female patients who would normally be considered asymptomatic carriers of the genetic mutation(s).

The genetic biomarkers for glucose-6-phosphate dehydrogenase (G6PD) deficiency provides a method for screening patients to determine patients who are candidates for developing severe disease (such as hemolytic anemia and neonatal jaundice) resulting from insufficient expression of the G6PD enzyme, opening the door to targeted preventive treatment. The method involves obtaining samples of DNA from a patient and sequencing the G6PD gene on the X chromosome. Identification of at least one haplotype selected from the group consisting of the Jeddah A, Jeddah B, Jeddah C, Jeddah D, and Jeddah E haplotypes is diagnostic for inadequate expression of G6PD enzyme to a degree that represents high risk of severe disease from deficiency of the enzyme. Haplotypes may be identified directly in male patients, who are hemizygous for the X chromosome, and may be identified by PHASE haplotype reconstruction in female patients,

The description that follows employs the following definitions:

“Encode.” A polynucleotide is said to “encode” or “code” for a polypeptide if, in its native state or when manipulated by methods well known to those skilled in the art, it can be transcribed and/or be translated to produce the mRNA for and/or the polypeptide or a fragment thereof.

“Gene.” A segment of DNA that contains all of the information for the regulated biosynthesis of an RNA product, including promoters, exons, introns, and other untranslated regions that control expression,

“Haplotype.” A member of a polymorphic set, e.g., a sequence of nucleotides found at one or more polymorphic sites in a locus in a single chromosome of an individual. In particular, as used herein, the nucleotides themselves need not be in sequence in the DNA molecule itself, but may be a set of single nucleotide polymorphisms that occur at different locations in a population being studied. The nucleotide locations are artificially or arbitrarily grouped together to form a haplotype sequence in order to study linkages or patterns in the mutations.

“Isolated.” An “isolated” nucleic acid (e.g., an RNA, DNA or a mixed polymer) is one that is substantially separated from other cellular components that naturally accompany a native human sequence or protein, e.g., ribosomes, polymerases, many other genome sequences and proteins. An isolated DNA molecule or DNA sequence is a sequence of nucleotides that has been chemically cleaved or physically separated from a native human sequence.

“Linkage Disequilibrium.” A non-random association of alleles at two or more loci. The term also refers to the case where the observed frequency of haplotypes in a population does not correspond to haplotype frequencies predicted by multiplying together the frequency of individual genetic markers in each haplotype.

“Locus.” A location on a chromosome or DNA molecule corresponding to a gene or a physical or phenotypic feature, and may be the location of a single nucleotide.

“Non-Conservative Mutation.” A mutation that results in an amino acid change that has different properties than the native amino acid. A “conservative mutation” is a mutation in a nucleotide that does not result in a change having different properties than the native amino acid, and may result in no change in the native amino acid due to degeneracy of the genetic code.

“Polymorphism.” The sequence variation observed in an individual at a polymorphic site. Polymorphisms include nucleotide substitutions, insertions, deletions and microsatellites and may, but need not, result in detectable differences in gene expression or protein function.

“Polynucleotide.” A nucleic acid molecule comprised of single-stranded RNA or DNA, or comprised of complementary, double-stranded DNA.

“Single nucleotide polymorphism”, or SNP, is a polymorphic change in a single nucleotide, rather than multiple nucleotides in sequence.

“X-linked mutation.” An X-linked mutation is a mutation in a gene located in the X-chromosome.

The present inventors sequenced the G6PD gene in male and female G6PD-deficient patients from the Saudi Arabian Population. They determined the patterns of mutation and polymorphism in cis (haplotypes) within the gene by physical linkage analysis in male patients, and by haplotype reconstruction in female patients. In addition, the correlation between specific G6PD haplotypes and the activity of GOD was examined in all male patients, and in female patients who were homozygous for a given haplotype. Through this process, the inventors discovered five novel haplotypes, Jeddah A, B, C, D, and E, each represented by combinations of two or three non-conservative amino acid substitutions.

In particular, 250 G6PD-deficient and non-deficient individuals (164 male, 86 female, among whom 182 were G6PD-deficient and 68 were normal) were studied, the individuals originating mostly from the western region of the Kingdom of Saudi Arabia. Patient ages ranged from newborn to 50 years. All of the patients were subjected to quantitative measurement of their G6PD enzyme to differentiate between their pathological states.

Genomic DNA was extracted from whole blood and quantitated using standard methods using Qiagen QIAamp DNA Blood Mini Kits. PCR primers were designed using Primer 3.0 software obtained from frodo.wi.mit.edu. A G6PD reference sequence (NT 167198.1) was used to identify intron-exon boundaries. PCR primers were designed to amplify exons (including associated intron-exon boundaries) for the entire coding region. The primer length was typically 18-22 nucleotides with a GC ratio of about 50%. The specificity of the PCR primers was ascertained by performing homology searches using the NCBI BLAST tool. Oligonucleotides were synthesized by Eurofins MWG Operon. Sequences of the PCR primers are shown in Table 1, below.

TABLE 1
PCR Primer Sequences

| | | Forward Primer | Reverse Primer | PCR | |
|---|---|---|---|---|---|
| Frag- | | sequence | sequence | product | |
| ment | Exon(s) | (5′-3′) | (5′-3′) | size | |
| 1 | 1, 2 | SEQ ID NO.: 1 | SEQ ID NO.: 2 | 1376 | bp |
| 2 | 3, 4, 5 | SEQ ID NO.: 3 | SEQ ID NO.: 4 | 1125 | bp |
| 3 | 6, 7 | SEQ ID NO.: 5 | SEQ ID NO.: 6 | 563 | bp |
| 4 | 8 | SEQ ID NO.: 7 | SEQ ID NO.: 8 | 202 | bp |
| 5 | 9, 10 | SEQ ID NO.: 9 | SEQ ID NO.: 10 | 741 | bp |
| 6 | 11, 12, 13 | SEQ ID NO.: 11 | SEQ ID NO.: 12 | 1151 | bp |

Touchdown PCR reactions contained genomic DNA (100 ng), PCR forward primer (10 pmol), PCR reverse primer (10 pmol), dNTPs (10 mM), 0.5U Hot Start Taq DNA polymerase (Qiagen, 5 U/μL), and 10× PCR buffer (Qiagen) containing 1.5 mM. MgCl₂ in a total volume of 25 μL. Samples were initially denatured at 95° C. for 15 minutes. DNA amplification was performed using 30 cycles of denaturation at 95° C. for 30 seconds, initial annealing started at 70° C. for 30 seconds, and extension at 72° C. for 30 seconds. The annealing temperature was reduced by 0.5° C. at each subsequent cycle. Another 30 cycles of fixed annealing temperature of PCR was performed, starting by denaturation at 95° C. for 30 seconds, annealing at 54° C. for 30 seconds and extension at 72° C. for 30 seconds. A final extension step at 72° C. for 5 minutes was performed to allow the newly synthesized fragments to complete replication. All PCRs were performed using a Bio-Rad DNA Thermal Cycler (Bio-Rad, USA).

PCR products were ethanol-precipitated and subjected to DNA sequencing using a BigDye Terminator v1.1./v3.1 Cycle Sequencing Kit (Applied Biosystems), and an Applied Biosystems automated DNA sequencer (ABI PRISM Genetic Analyzer 3130, Hitachi).

Patient genotypes were assigned using BLAST alignment with G6PD reference genomic DNA sequence NT 167198 (UCSC Genome Browser, March 2006 Build). All sequences matched the reference genomic sequence except for mutation or polymorphism sites at the loci shown in Table 2, below. Haplotypes in male patients were evident, since males are hemizygous for the X chromosome. Haplotypes in female patients were reconstructed using maximum likelihood analysis (PHASE version 2.1), as taught by Stephens et al., A New Statistical Method For Haplotype Reconstruction From Population Data, American Journal of Human Genetics 68:978-989 (2001), and Stephens M & Scheed P, Accounting For Decay of Linkage Disequilibrium in Haplotype Inference and Missing-Data Imputation, American Journal of Human Genetics 76: 449-462 (2005), with allowance for recombination and decay of linkage disequilibrium with distance.

TABLE 2
Mutations and polymorphisms identified in patients

| | | | | Amino acid | | Frequency | | |
|---|---|---|---|---|---|---|---|---|
| | Exon/intron | Nucleotide | Nucleotide | number and | | F (N) | | M (N) |
| Locus^{a} | number | number^{b} | mutation | substitution | Designation | Hom. | Het. | Hemi. |
| 0 | Exon 3 | 153417565 | T > C | p.Ile48Thr | AURES | 13 (86) | 20 (86) | 58 (163) |
| 1 | Exon 4 | 153417411 | G > A | p.Val68Met | A- | 4 (86) | 8 (86) | 20 (163) |
| 2 | Exon 5 | 153416686 | A > G | p.Asn126Asp | A- | 0 (86) | 8 (86) | 23 (163) |
| 3 | Exon 6 | 153415828 | C > T | p.Ser188Phe | MEDITERRANEAN | 9 (86) | 20 (86) | 36 (163) |
| 4 | Exon 6 | 153415757 | A > G | p.Met212Val | SIBARI | 0 (86) | 0 (86) | 2 (163) |
| 5 | Exon 9 | 153414531 | G > A | p.Val291Met | VIANGCHAN | 0 (86) | 0 (86) | 1 (163) |
| 6 | Exon 9 | 153414434 | T > C | p.Leu323Pro | A- | 0 (86) | 1 (86) | 0 (163) |
| 7 | Exon 11 | 153413848 | C > T | p.Tyr437Tyr | — | 14 (61) | 18 (61) | 51 (102) |
| 8 | Intron 11 | 153413702 | T > C | — | — | 32 (61) | 18 (61) | 77 (102) |
| 9 | Exon 12 | 153413666 | G > A | p.Arg463His | ANANT | 1 (61) | 0 (61) | 3 (102) |
| 10 | Exon 12 | 153413623 | C > T | p.Pro477Pro | — | 0 (61) | 1 (61) | 1 (102) |
| 11 | Intron 13 | 153413340 | —/GGA | — | — | 0 (61) | 2 (61) | 1 (102) |
| 12 | Intron 13 | 153413052 | A > G | — | — | 1 (61) | 3 (61) | 1 (102) |

^{a}Locus designation used herein, and for linkage disequilibrium locus codes
^{b}Nucleotide numbers are from G6PD reference sequence (UCSC Genome Browser, March 2006 build)

Whole blood was used to quantitatively measure G6PD enzyme activity using a UDICHEM-310 spectrophotometer (United Diagnostic Industry, Damman, K. S. A). G6PD activity was determined for all samples according to WHO recommendations using the UV/Kinetic method (United Diagnostics Industry G6PD quantitative kit 038-020, UDI, Dammam, K. S. A.).

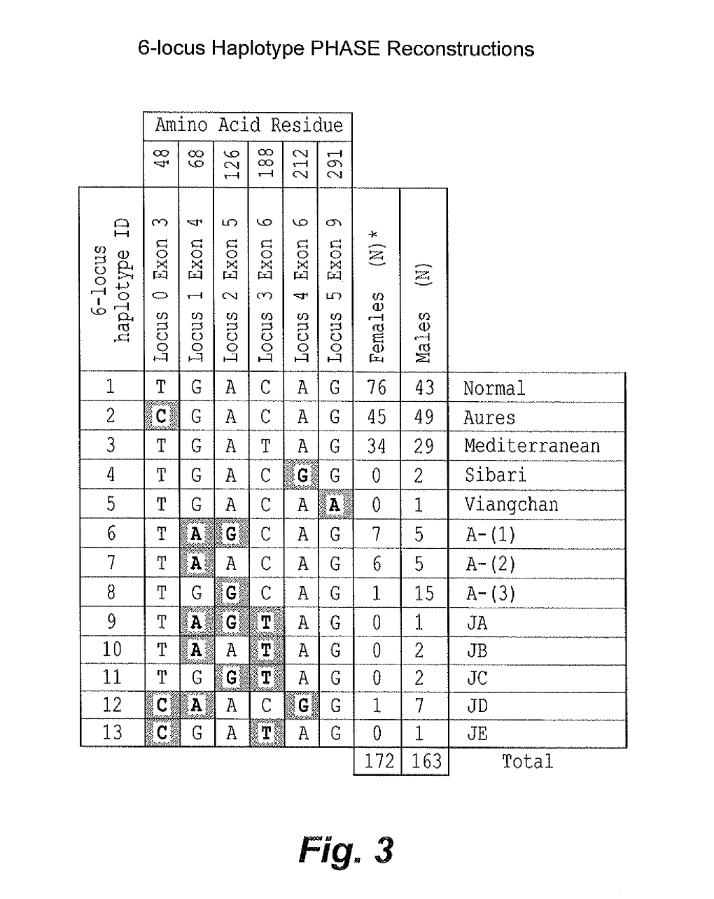

Mutations in the G6PD gene tend to be point locations, i.e., mutations of a single nucleotide, referred to herein as a single nucleotide polymorphism. Occasionally, a GOD gene will exhibit mutations in more than one nucleotide, but the nucleotides are not usually adjacent to each other or in sequence, and may even be in different exons or in an intron. Results from our study of the 250 Saudi individuals are represented in Table 2 and in the table of FIG. 3. In FIG. 3, non-conservative mutated bases are shown highlighted by a gray background. Mutant loci are numbered from left to right and correspond to loci 0-5, as designated in Table 2. Totals represent haplotype numbers, not patient numbers (except in males, where these are identical). A-(1), A-(2), and A-(3) are variants of the A-phenotype. The column labeled “Females (N)*” provides the haplotype count. Although some applications, e.g., DNA testing for paternity, may use a form of multi-locus sequencing, each locus in such DNA tests is composed of a variable number of bases in short sequences. By contrast, each locus in the locus column of FIG. 3 refers to the site of a single nucleotide, which is identified by the “nucleotide number” corresponding to the locus in Table 2, above, the nucleotide number corresponding to the genomic reference sequence for G6PD from the UCSC (University of California Santa Cruz) Genomic Browser, March 2006 assembly or build. The table in FIG. 3 shows the nucleotide base at the corresponding nucleotide position in a format that is sometimes referred to herein as a haplotype sequence, or simply haplotyping, although the bases do not naturally occur in sequence in the gene and have not been artificially placed in sequence physically by the inventors.

Generally, G6P1) haplotypes were identified directly in males because males are hemizygous for the X chromosome, and therefore genotypes at each G6PD locus are in physical linkage.

Referring to FIG. 3, 6-locus haplotyping was performed for 163 male patients and revealed 13 haplotypes, One haplotype was ‘normal,’ that is, possessed no amino acid substitutions in the sequenced region associated with G6PD deficiency. This ‘normal’ haplotype was observed in 43 male patients (26.38%). In males, the three most common pathogenic 6-locus haplotypes observed in order of frequency were Aures, characterized by a single p.IIe48Thr mutation (49/163, 30.06%); Mediterranean, characterized by a single p.Ser188Phe mutation (29/163, 17.79%); and variants of A- designated A-(1-3), characterized by a combination of p.Val68Met and p.Asn126Asp mutations, or by one or the other mutation, respectively (25/163, 15.33%). Other haplotypes were each present in less than 2% of male patients. Five of these haplotypes were previously unreported (Jeddah A, B, C, D and E), and all five were characterized by novel combinations of two or three non-conservative amino acid substitutions, as shown in Table 3. These novel haplotypes accounted for 13/163 (7.97%) of the male patients. Jeddah D was the most common of the five novel haplotypes (detected in 7/163 (429%) of male patients).

TABLE 3
Novel haplotypes identified in Jeddah patients

| Haplotype | Amino acid changes |
|---|---|
| Jeddah A | Val68Met + Asn126Asp + Ser188Phe |
| Jeddah B | Val68Met + Ser188Phe |
| Jeddah C | Asn126 + Ser188Phe |
| Jeddah D | Ile48Thr + Val68Met |
| Jeddah E | Ile48Thr + Ser188Phe |

In particular, from Tables 2 and 3 and FIG. 3, the Jeddah A haplotype is characterized by a mutation in the base at nucleotide number 153417411 from guanine to adenine, which results in a non-conservative amino acid change in the G6PD enzyme or protein from Valine to Methionine at amino acid residue number 68, and a second mutation at nucleotide number 153416686 from adenine to guanine, which results in a non-conservative amino acid change in the G6PD enzyme or protein from Asparagine to Aspartic Acid at amino acid residue number 126, and a third mutation at nucleotide number 153415828 from cytosine to thymine, which results in a non-conservative amino acid change in the G6PD enzyme or protein from Serine to Phenylalanine at amino acid residue number 188.

The Jeddah B haplotype is characterized by a mutation in the base at nucleotide number 153417411 from guanine to adenine, which results in a non-conservative amino acid change in the G6PD enzyme or protein from Valine to Methionine at amino acid residue number 68, and a second mutation at nucleotide number 153415828 from cytosine to thymine, which results in a non-conservative amino acid change in the G6PD enzyme or protein from Serine to Phenylalanine at amino acid residue number 188.

The Jeddah C haplotype is characterized by a mutation in the base at nucleotide number 153416686 from adenine to guanine, which results in a non-conservative amino acid change in the G6PD enzyme or protein from Asparagine to Aspartic acid at amino acid residue number 126, and a second mutation at nucleotide number 153415828 from cytosine to thymine, which results in a non-conservative amino acid change in the G6PD enzyme or protein from Serine to Phenylalanine at amino acid residue number 188.

The Jeddah D haplotype is characterized by a mutation in the base at nucleotide number 153417565 from thymine to cytosine, which results in a non-conservative amino acid change in the G6PD enzyme or protein from Isoleucine to Threonine at amino acid residue number 48, and a second mutation at nucleotide number 153417411 from guanine to adenine, which results in a non-conservative amino acid change in the G6PD enzyme or protein from Valine to Methionine at amino acid residue number 68.

The Jeddah E haplotype is characterized by a mutation in the base at nucleotide number 153417565 from thymine to cytosine, which results in a non-conservative amino acid change in the G6PD enzyme or protein from Isoleucine to Threonine at amino acid residue number 48, and a second mutation at nucleotide number 153415828 from cytosine to thymine, which results in a non-conservative amino acid change in the G6PD enzyme or protein from Serine to Phenylalanine at amino acid residue number 188.

G6PD haplotypes were identified in female patients using PHASE haplotype reconstruction. As shown in FIG. 3, 6-locus haplotyping was performed for 86 female patients (172 haplotypes) and revealed 8 haplotypes. In females, the two most common pathogenic 6-locus haplotypes observed in order of frequency were Aures (45/172, 26.16%) and Mediterranean (34/172, 19.76%). Other pathogenic haplotypes were each present with a haplotype frequency less than 5%. Only 2 of the novel Jeddah haplotypes were identified in the female patients (Jeddah 13 and Jeddah D).

For both the male and the female patient cohorts, no individual novel non-conservative mutation sites were identified. Rather, the 5 novel haplotypes (Jeddah A-E) were defined by previously unreported combinations of extant non-conservative mutations. Jeddah B and D were seen in both males and females, either by physical linkage or haplotype reconstruction, respectively. Jeddah A, C, and E were only observed in males.

Haplotype data permitted the analysis of linkage disequilibrium (LD) between pairs of adjacent mutant or polymorphic loci beyond the 6-loci used for haplotype analysis. The synonymous p.Tyr437Tyr and the T>C polymorphism in intron 11 exhibit a LOD score of 9.89 and D′ value of 0.949. Significant linkage disequilibrium exists between the p.Tyr437Tyr polymorphism and the p.Asn126Asp mutation (LOD=2.43, D′=0.765) or the p.Va168Met mutation (LOD=2.92, D′=0.725). Significant linkage disequilibrium is also detectable between p.Ser188Phe and p.Ile48Thr (LOD=2.82, D′=1.0) and pAsn126Asp with p.Va168Met (LOD=20.95, D′=0.805), suggesting that several other haplotypes can be detected if the synonymous changes or intronic polymorphisms were to be included.

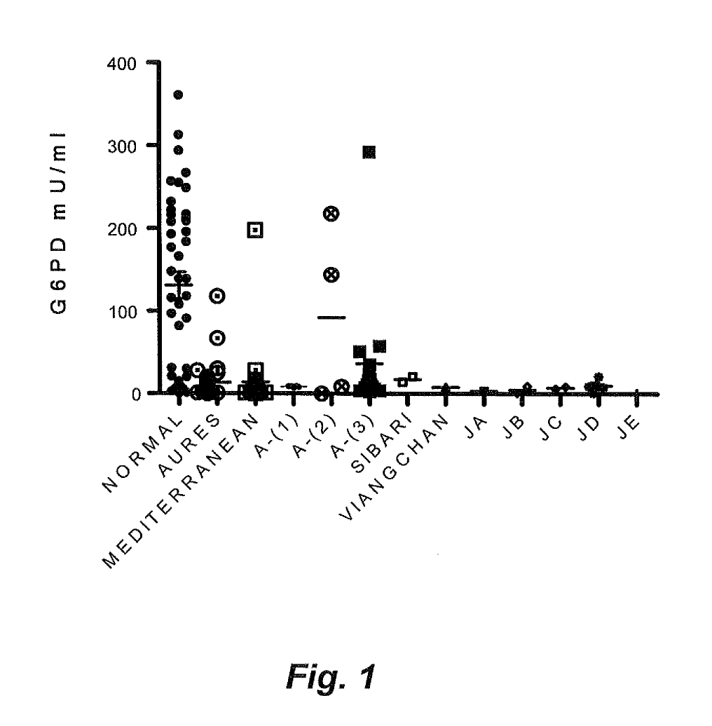

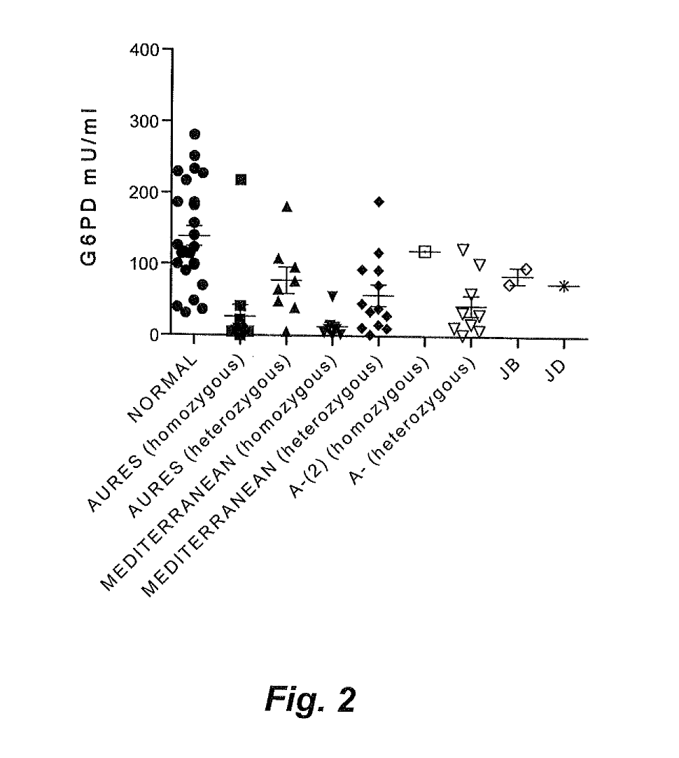

FIGS. 1-2 and Tables 4-5 illustrate the effect of different G6PD haplotypes on the level of G6PD expressed. The dominant effect on expression of a given haplotype could be assessed by analyzing males who are hemizygous and females who are homozygous for that haplotype, since these individuals only possess the mutant haplotype. FIG. 1 shows results for haplotypes in males representing non-conservative amino acid mutations, where expression of the haplotype is dominant. FIG. 2 shows results for haplotypes in females representing non-conservative amino acid mutations, where expression of the haplotype is mosaic, Mosaic expression of G6PD on the affected X chromosome due to skewed X-inactivation can lead to clinical G6PD deficiency.

TABLE 4
G6PD enzyme values according to haplotype in male patients

| | | | | | | Std. |
|---|---|---|---|---|---|---|
| | Number | Min. | Max. | Median | Mean | Error |
| Normal | 43 | 0.90 | 361.00 | 139.00 | 131.30 | 16.01 |
| Aures | 49 | 0.28 | 118.20 | 9.00 | 13.29 | 2.67 |
| Med. | 29 | 0.20 | 198.00 | 6.86 | 13.83 | 6.68 |
| A-(1) | 5 | 7.00 | 9.00 | 9.00 | 8.40 | 0.40 |
| A-(2) | 4 | 0.25 | 218.00 | 76.20 | 92.66 | 53.22 |
| A-(3) | 15 | 2.33 | 292.70 | 11.00 | 36.52 | 18.83 |
| Sibari | 2 | 14.00 | 20.84 | 17.42 | 17.42 | 3.42 |
| Viang. | 1 | 7.96 | 7.96 | 7.96 | 7.96 | 0.00 |
| Jeddah A | 1 | 3.00 | 3.00 | 3.00 | 3.00 | 0.00 |
| Jeddah B | 2 | 0.82 | 9.00 | 4.91 | 4.91 | 4.09 |
| Jeddah C | 2 | 6.31 | 8.43 | 7.37 | 7.37 | 1.06 |
| Jeddah D | 7 | 0.90 | 21.00 | 9.00 | 9.87 | 2.24 |
| Jeddah E | 1 | 1.15 | 1.15 | 1.15 | 1.15 | 0.00 |

TABLE 5
G6PD enzyme values according to haplotype in female patients

| | | | | | | Std. |
|---|---|---|---|---|---|---|
| | Number | Min. | Max. | Median | Mean | Error |
| Normal | 26 | 31 | 281 | 120 | 138.9 | 13.87 |
| Aures | 13 | 0018 | 218 | 9 | 26.62 | 16.21 |
| (hom.) | | | | | | |
| Aures | 8 | 5.9 | 181.6 | 70.77 | 77.5 | 18.77 |
| (het.) | | | | | | |
| Med. | 8 | 0.939 | 55 | 9.05 | 13.16 | 6.193 |
| (hom.) | | | | | | |
| Med. | 13 | 1.9 | 189.4 | 38.54 | 57.53 | 14.91 |
| (het.) | | | | | | |
| A-(2) | 1 | 120 | 120 | 120 | 120 | 0 |
| A- | 9 | 1.8 | 123 | 30 | 42.98 | 14.43 |
| Jeddah B | 2 | 74 | 97.17 | 85.58 | 85.58 | 11.58 |
| Jeddah D | 1 | 74 | 74 | 74 | 74 | 0 |

As noted above, G6PD is one cause of neonatal jaundice, apparently because the deficiency in the enzyme results in an excessive accumulation of bilirubin in the blood of the newborn. Left untreated, neonatal jaundice may result in the development of kernicterus, which can leave the child with mental retardation. Fortunately, neonatal jaundice can be treated and kernicterus may be avoided if the jaundice is diagnosed timely. The genetic biomarkers described herein may assist in such treatment. First, adults who have been screened by the DNA testing described above and identified as exhibiting the Jeddah A, B, C, D, or B haplotype may be educated that their children are at high risk for suffering from G6PD deficiency and educated in the signs and symptoms of neonatal jaundice, and of the importance of seeking immediate medical attention for the condition, resulting in early diagnosis. Second, upon appearance of symptoms of neonatal jaundice, a sample of DNA may be obtained from the newborn and sequenced as described above. Identification of the Jeddah A, B, C, D, or E haplotype would confirm severe G6PD deficiency as the probable cause of the jaundice, and the need for appropriate treatment to avoid the development of kernicterus.

Patients with G6PD deficiency also may suffer from hemolytic anemia, which may be acute or chronic. Acute hemolytic anemia usually manifests itself after ingestion of fava beans or certain drugs, or as a complication of infection. Episodes of acute hemolytic anemia may resolve without treatment, since the body continues to produce red blood, although monitoring for possible blood transfusions and renal failure may be necessary. If the deficiency in G6PD enzyme is severe enough, as it may well be when the Jeddah A, B, C, D, or E haplotypes are present, the hemolytic anemia may turn out to be chronic. In this event, folic acid supplements, red cell blood transfusions, and iron chelators may be necessary. In addition, patients with chronic hemolytic anemia may be more susceptible to acute attacks of hemolysis, since these patients experience acute attacks upon ingestion of a wider range of drugs and at lower dosages. The genetic biomarkers described above may assist in treatment for hemolytic anemia by suggesting the need for educating patients to avoid the ingestion of fava beans, educating the patient on which drugs to avoid, and in the case of chronic hemolytic anemia, suggesting the need for genetic counseling and prenatal diagnosis when the patient is identified as exhibiting the Jeddah A through E haplotypes.

It will be noted that the normal tests for G6PD deficiency, such as the fluorescent spot test and the quantitative spectrophotometric assay for G6PD activity, are unreliable for heterozygous females. However, the present inventor's study shows the Jeddah A through Jeddah E haplotypes can be detected in heterozygous females, and that the presence of one of these five haplotypes indicates a high risk of severe G6PD deficiency, so that when a heterozygous female experiences an episode of acute hemolytic anemia and also tests positive for the presence of one of the five haplotypes, G6PD is implicated as the cause for the episode.

It will also be understood that the Jeddah A, Jeddah B, Jeddah C, Jeddah D, and Jeddah E haplotypes are not the only haplotypes that involve more than one non-conservative amino acid change in the G6PD protein. However, the present study shows that when Jeddah A, B, C, D, or E is present, the patient is at risk for a severe deficiency in the G6PD enzyme. It will also be understood that the presence of the Jeddah A, B, C, D, or E haplotype may also be determined by detecting the sequence of amino acid residues in the G6PD monomer for the presence of the pairs of amino acid changes listed in Table 3.

A method of predicting a patient at high risk of glucose-6-phosphate dehydrogenase (G6PD) deficiency may comprise the steps of obtaining a DNA sample from the patient (which may be a tissue sample or a fluid sample, such as blood or saliva); testing the DNA sample for the presence of a haplotype selected from the group consisting of Jeddah A, Jeddah B, Jeddah C, Jeddah D, and Jeddah h, wherein the Jeddah A haplotype comprises the base adenine at nucleotide number 153417411, the base guanine at nucleotide number 153416686, and the base thymine at nucleotide number 153415828, the Jeddah B haplotype comprises the base adenine at nucleotide number 153417411 and the base thymine at nucleotide number 153415828, the Jeddah C haplotype comprises the base guanine at nucleotide number 153416686 and the base thymine at nucleotide number 153415828, the Jeddah D haplotype comprises the base cytosine at nucleotide number 153417565 and the base adenine at nucleotide number 153417411, and the Jeddah E haplotype comprises the base cytosine at nucleotide number 153417565 and the base thymine at nucleotide number 153415828; and determining that the patient is at risk of developing severe G6PD deficiency when a haplotype selected from the group consisting of Jeddah A, Jeddah B, Jeddah C, Jeddah D, and Jeddah E is detected. The step of detecting the haplotype may include the steps of replicating fragments of the DNA sample by polymerase chain reaction (PCR) and sequencing the replicated fragments by an automated DNA sequencing machine, by fluorescent markers and gel electrophoresis, or by any other known method, including forming probes of complementary DNA from known exemplars of the haplotypes and matching with the samples, etc.

It is to be understood that the present invention is not limited to the embodiments described above, but encompasses any and all embodiments within the scope of the following claims.

1. A method of predicting that a patient is at a high risk of developing glucose-6-phosphate dehydrogenase (G6PD) deficiency symptoms, comprising the steps of:
obtaining a DNA sample from the patient;
testing the DNA sample for the presence of a haplotype selected from the group consisting of Jeddah A, Jeddah B, Jeddah C, Jeddah D, and Jeddah E in the patient's human G6PD gene, wherein:
the Jeddah A haplotype comprises the base adenine at nucleotide number 153417411, the base guanine at nucleotide number 153416686, and the base thymine at nucleotide number 153415828;
the Jeddah B haplotype comprises the base adenine at nucleotide number 153417411 and the base thymine at nucleotide number 153415828;
the Jeddah C haplotype comprises the base guanine at nucleotide number 153416686 and the base thymine at nucleotide number 153415828;
the Jeddah D haplotype comprises the base cytosine at nucleotide number 153417565 and the base adenine at nucleotide number 153417411, which bases comprise SEQ ID NO. 3 and SEQ ID NO 4; and
the Jeddah E haplotype comprises the base cytosine at nucleotide number 153417565 and the base thymine at nucleotide number 153415828; and
wherein said step of testing the DNA sample for the presence of a haplotype further comprises the steps of:
replicating fragments of the patient's G6PD gene by polymerase chain reaction (PCR); and
sequencing the fragments by automatic DNA sequencing machine;
wherein said step of replicating fragments includes using a forward primer having the sequence consisting of SEQ ID NO. 3 and a reverse primer having the sequence consisting of SEQ ID NO. 4 to replicate the fragments by polymerase chain reaction (PCR);
predicting that the patient is at a high risk of developing G6PD deficiency symptoms when a haplotype selected from the group consisting of Jeddah A, Jeddah B, Jeddah C, Jeddah D, and Jeddah E is detected.

2. The method of predicting that a patient is at a high risk of developing glucose-6-phosphate dehydrogenase (G6PD) deficiency symptoms according to claim 1, wherein said step of obtaining a DNA sample comprises obtaining a sample of the patient's tissue.

3. The method of predicting that a patient is at a high risk of developing glucose-6-phosphate dehydrogenase (G6PD) deficiency symptoms according to claim 1, wherein said step of obtaining a DNA sample comprises obtaining a sample of the patient's blood.

4. The method of predicting that a patient is at a high risk of developing glucose-6-phosphate dehydrogenase (G6PD) deficiency symptoms according to claim 1, wherein said step of obtaining a DNA sample comprises obtaining a sample of the patient's saliva.

5.-18. (canceled)


SEQUENCE LISTING (12 sequences)

SEQ ID NO: 1 - DNA, 18 bp, Artificial Sequence
  forward primer for exons 1,2 of G6PD gene
caatagggccggcttgac

SEQ ID NO: 2 - DNA, 22 bp, Artificial Sequence
  reverse primer for exons 1,2 of G6PD gene
tgcaacaattagttggaaaagc

SEQ ID NO: 3 - DNA, 20 bp, Artificial Sequence
  forward primer for exons 3,4,5 of G6PD gene
tgtccccagccacttctaac

SEQ ID NO: 4 - DNA, 20 bp, Artificial Sequence
  reverse primer for exons 3,4,5 of G6PD gene
ctcatagagtggtgggagca

SEQ ID NO: 5 - DNA, 19 bp, Artificial Sequence
  forward primer for exons 6,7 of G6PD gene
aggggttcaagggggtaac

SEQ ID NO: 6 - DNA, 18 bp, Artificial Sequence
  reverse primer for exons 6,7 of G6PD gene
tgcagggtgactggctct

SEQ ID NO: 7 - DNA, 18 bp, Artificial Sequence
  forward primer for exon 8 of G6PD gene
acagggcggggagctaag

SEQ ID NO: 8 - DNA, 18 bp, Artificial Sequence
  reverse primer for exon 8 of G6PD gene
gtgcctcgtcacagatgg

SEQ ID NO: 9 - DNA, 18 bp, Artificial Sequence
  forward primer for exons 9,10 of G6PD gene
cctgagggctgcacatct

SEQ ID NO: 10 - DNA, 20 bp, Artificial Sequence
  reverse primer for exons 9,10 of G6PD gene
gtgtcttgctgatgccactg

SEQ ID NO: 11 - DNA, 20 bp, Artificial Sequence
  forward primer for exons 11,12,13 of G6PD gene
tggcatcagcaagacactct

SEQ ID NO: 12 - DNA, 20 bp, Artificial Sequence
  reverse primer for exons 11,12,13 of G6PD
gacaaggaagtgggtcctca